(19) United States
(12) Patent Application Publication
(10) Pub. No.: US 2011/0270127 A1
(43) Pub. Date: Nov. 3, 2011

(54) Biological Fluid Receptacle

(76) Inventors: Shany Vered, Ramat Gan (IL); Isaac Tavori, Ramat Gan (IL)

(21) Appl. No.: 12/311,049

(22) PCT Filed: Sep. 18, 2007
(86) PCT No.: PCT/IL07/01144

§ 371 Date: Jul. 19, 2011

Related U.S. Application Data
(60) Provisional application No. 60/845,173, filed on Sep. 18, 2006.

(51) Int. Cl. A61B 5/00

(52) U.S. Cl. 600/573

(57) ABSTRACT

There is provided a biological fluid receptacle that includes an outer surface adapted to be affixed to a carrier and a receptacle cavity adapted to receive and retain biological fluids.

### BACKGROUND

Conception may normally be achieved within twelve months in 80-85% of couples who use no contraceptive measures. Approximately 15% of couples attempting their first pregnancy meet with failure. Couples are defined as primarily infertile if they have been unable to achieve a pregnancy after one year of unprotected intercourse, in which case they should be evaluated for infertility. Although men are responsible for more than 50% of infertility cases, the social stigma associated with male sexual issues has created a general reluctance of men to seek a physician's diagnosis.

Data available over the past twenty years reveal that in approximately 30% of cases pathology is found in the man alone, and in another 30% both the man and woman are abnormal. Therefore, the male factor is at least partly responsible in about 50% of infertile couples. Important issues related to the evaluation of the male factor include the most appropriate time for the male evaluation, the most efficient format for a comprehensive male exam, and definition of rationale and effective behavior, medical and surgical regimens in the treatment of these disorders.

It is important in the evaluation of infertility to consider the couple as a unit in evaluation and treatment and to proceed in a parallel investigative manner until a problem is uncovered. It has been shown that the longer a couple remains subfertile, the worse their chance for an effective cure. Many couples experience significant apprehension and anxiety after only a few months of failure to conceive. Unduly prolonged unprotected intercourse should not be advocated before a workup of the man is instituted. Initial screening of the man should be considered whenever the patient presents with the chief complaint of infertility. This initial evaluation should be rapid, non-invasive and cost effective.

Spermatogenesis is a complex process whereby primitive stem cells or spermatogonia either divide to reproduce themselves for stem cell renewal or they divide to produce daughter cells that will later become spermatocytes. The spermatocytes eventually divide, go through maturation process, and give rise to spermatids. The spermatids then undergo a transformation into spermatozoa which is the matured form of sperm cells. This transformation includes nuclear condensation, acrosome formation, loss of most of the cytoplasm, development of a tail and arrangement of the mitochondria into the middle piece of the sperm cell, which basically becomes the engine room to power the tail. Groups of germ cells tend to develop and pass through spermatogenesis together. This sequence of developing germ cells is called a generation. These generations of germ cells are basically in the same stage of development. There are six stages of seminiferous epithelium development: the progression from stage one through stage six constitutes one cycle. In humans, the duration of each cycle is approximately 16±1 days and 4 cycles are required for a mature sperm cell to develop from early spermatogonia. Therefore, the duration of the entire spermatogenic cycle in humans is 4 cycles times 16 days equals 64±4.5 days (Junqueira Junqueira L C and Carneiro J (1983): Basic histology. 4^{th} edition. Lange medical publications, California, (incorporated by reference)).

During emission, secretions from the seminal vesicles and prostate are deposited into the posterior urethra. Prior to ejaculation, peristalsis of the vas deferens and bladder neck occur under sympathetic nervous control. During ejaculation, the bladder neck tightens and the external sphincter relaxes with the semen being propelled through the urethra via rhythmic contractions of the perineal and bulbourethral muscles. The major volume of the seminal fluid comes from the seminal vesicles and secondarily the prostate. The seminal vesicles provide the nourishing substrate fructose as well as prostaglandins and coagulating substrates.

A recognized function of the seminal plasma is its buffering effect on the acidic vaginal environment. The coagulum formed by the ejaculated semen liquefies within 20 to 30 minutes (externally to the woman's body) as a result of prostatic proteolytic enzymes. The prostate also adds zinc, phospholipids, spermine and phosphatase to the seminal fluid. The first portion of the ejaculate characteristically contains most of the prostatic secretions and seminal vesicle secretions, while the second portion is composed of most of the spermatozoa.

Fertilization normally takes place within the uterine tubes after ovulation has occurred. During the menstrual mid cycle, the cervical mucus changes to become more abundant, thinner and more watery. These changes serve to facilitate entry of the sperm cells into the uterus and to protect the sperm cells from the highly acidic vaginal secretions. Physiologic changes in the spermatozoa, known as capacitation, occur within the female reproductive tract in order for fertilization to occur. As the sperm cell interacts with the egg, there is initiation of new flagellar movement called hyperactive motility and morphologic changes in the sperm cells that result in the release of proteolytic enzymes and exposure of parts of the sperm cells' structure, known as the acrosome reaction. As a result of these changes, the fertilizing sperm cell is able to reach the oocyte, traverse its various layers, and become incbrporated into the ooplasm.

Sperm cells counts have dropped by half in the last 50 years, and modern men have 20 percent less semen volume than their fathers. A report from researchers in Aberdeen suggested that the sperm cells concentration of men seen in their clinic had declined by 29% over the past 14 years. (British Fertility Society; 5, Jan. 2004, (incorporated by reference)). Decline in sperm cells count may be due to many factors such as exposure to common chemicals like alcohol, pesticides in food etc. Damage to sperm cells caused by lifestyle risk factors known to decrease sperm cells quality includes cigarette smoking, chronic stress and nutritional deficiencies. Other reasons for infertility include congenital/inherited factors, and health conditions like prostatitis and diabetes that may also affect sperm cells production.

Semen analysis is the cornerstone of infertility investigation in the male. On the basis of the result, a couple is provided with prognostic and diagnostic information to assist in their management.

Male infertility is very common. About one in twenty men is sub fertile and a male factor is present in half of all infertile couples. About one third of all IVF (In Vitro Fertilization) procedures are performed for male factor infertility. Most infertile men produce low numbers of sperm cells, which may also show both poor swimming ability (motility) and be abnormally shaped. In such men, only a small number of normally shaped motile sperm cells are likely to swim up the woman's fallopian tube into the vicinity of the egg and even then may be unable to fertilise the egg.

The most widely test used is the semen analysis, which requires a great deal of expertise to comply with the high standards prescribed by the World Health Organization (WHO, 1999) (WHO Laboratory Manual for Examination of human semen and Semen-Cervical Mucus Interaction. Cambridge University Press, Cambridge, UK, incorporated by reference). According to the WHO standards, examination of human semen include the following: semen appearance, semen volume, semen viscosity, semen pH, sperm cells concentration, sperm cells motility, cellular elements other than spermatozoa, sperm cells agglutination, sperm cells vitality, sperm cells morphology, antibody-coating of spermatozoa, hypo-osmotic swelling test, semen culture, biochemical assays for accessory sex organ function, concentrations of zinc, fructose and neutral α-glucosidase, computer-aided sperm cells analysis, zona-free hamster oocyte test, reactive oxygen species, human zona pellucida binding test, acrosome reaction, anti oxidants and trace elements.

Semen quality varies widely between men. Even for a particular man, a minimum of two sperm cells counts at least three weeks apart need to be taken to give a true indication of his sperm cells quality. The test is performed after 3-8 days of sexual abstinence. Ideally the laboratory provides an appropriate collection room in order to avoid changes in temperature during the transport of samples to the laboratory. A sperm cells count greater than 20 million/ml is considered normal, however the average for the population is about 60 million and some men have a sperm cells count of above 200 million/ml. Sperm cells counts between five and 20 million/ml do not necessarily indicate a severe infertility problem. The ability of sperm cells to swim is termed motility. Normally, greater than fifty percent of sperm cells show some motility. Markedly reduced nibtility problems can greatly reduce fertility. The sperm cells shape, termed morphology, is an important predictor of fertility and the accurate assessment of this feature requires great skill (De Sutter P Best Pract. Res. Clin. Obstet Gynaecol. 2006 Jun. 9, E-pub (Incorporated by reference).

Many men find the production of a semen sample an embarrassing and stressful experience. In addition, there is often a significant waiting time for an initial appointment and an additional delay before the results are available. All these factors heighten the anxiety associated with infertility investigations.

“FertilMarQ”, a diagnostic home kit for semen analysis measures a factor of sperm cells count. The test indicates sperm cells concentration in the semen sample.

Björndahl, L. et al. (Development of a novel home sperm test, Human reproduction, 21 (1): 145, 2006) developed a home sperm cells test to allow the patient to obtain an assessment of fertility potential at his convenience.

Some of the downsides of standard semen analysis as performed today, may include the following: most of the males are reluctant to perform semen analysis test; The information gathered during the semen analysis depends on proper collection of the specimen, which may be flawed when the male is stressed; Test quality is dependent on the skills of the technician or physician performing the test. Only about 5% of the world clinics meet all WHO standards for assessing the size and shape of sperm cells (Human Reproduction 2005 20(12):3441-3445). In addition, the sample may be exposed to environmental factors such as air (oxidation) and temperature.

In addition to the standard laboratory analysis, a post-coital test (PCT) may be performed separately by a gynecologist. The use of the PCT in the basic fertility work-up has been subject to debate over the last 10 years. The PCT traditionally is used as a test to diagnose cervical factor subfertility. An abnormal PCT in the presence of normal semen is considered to reflect cervical hostility. The routine use of the PCT has been reported to lead to more treatment without an associated increase in pregnancy rates.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

According to some embodiments, there is provided a biological fluid sample collector that includes an outer surface adapted to be affixed to a carrier and comprising at least two portions with different porosity characteristics; and a sample collector cavity adapted to receive biological fluids.

According to some embodiments, the carrier to which the outer surface of the biological fluid sample collector is affixed to may include a contraceptive device, a swab, an applicator, tweezers, tampon, a sampling cup and the like.

According to some embodiments, the biological fluids collected in the biological fluid sample collector may be selected from a group that includes semen, seminal fluid, cervical fluid, cervical mucus, vaginal fluid, mammalian cells, microorganisms, proteins, carbohydrates, ions, hormones, and the like, or any combination thereof.

According to further embodiments, at least one outer surface portion with different porosity may include a membrane.

According to some embodiments, the biological fluid sample collector cavity may further include inner compartments.

According to additional embodiments, the biological fluid sample collector may be used intrabody, extrabody or any combination thereof.

According to further embodiments, the biological fluids collected in the biological fluid sample collector may further be manipulated. Manipulation may include testing, sorting, interacting, diagnosing and the like, or any combination thereof. Manipulation may be performed intrabody, extrabody or both. Manipulation may further be performed within the biological fluid sample collector cavity, externally to the biological fluid sample collector cavity, or both.

According to some embodiments, there is provided a biological fluid receptacle that includes an outer surface adapted to be affixed to a contraceptive device and comprising at least two portions with different porosity characteristics and a receptacle cavity adapted to receive biological fluids.

According to some embodiments, the biological fluids received by the biological fluid receptacle may be selected from a group that includes semen, seminal fluid, cervical fluid, cervical mucus, vaginal fluid, mammalian cells, microorganisms, proteins, carbohydrates, ions, hormones and the like and any combination thereof.

According to some embodiments, the contraceptive device to which the outer surface of the receptacle is affixed to may be selected from a group that includes male condom, sponge, diaphragm, cervical cup, female condom or any combination thereof.

According to some embodiments, at least one outer surface portion with different porosity may include a membrane.

According to some embodiments, the receptacle cavity may further include inner compartments.

According to additional embodiments, the biological fluid receptacle of may be used intra body, extrabody or any combination thereof.

According to some embodiments, the biological fluids may be further manipulated. Manipulations may include testing, sorting, interacting, diagnosing and the like, or any combination thereof. Manipulations may be performed intrabody, extrabody or both. Manipulations may further be performed within the receptacle cavity, externally to the receptacle cavity, or both.

According to some embodiments there is provided a biological fluid receptacle that includes an outer surface integral with a contraceptive device and a receptacle cavity adapted to receive biological fluids.

According to some embodiments, the biological fluid may be selected from a group that includes semen, seminal fluid, cervical fluid, cervical mucus, vaginal fluid, mammalian cells, microorganisms, proteins, carbohydrates, ions, hormones, and the like, or any combination thereof.

According to some embodiments, the contraceptive device to which the outer surface of the receptacle is affixed to may be selected from a group that includes male condom, sponge, diaphragm, cervical cup, female condom or any combination thereof.

According to additional embodiments, the biological fluid receptacle may be used intra body, extrabody or any combination thereof.

According to some embodiments, the biological fluids may be further manipulated. Manipulations may include testing, sorting, interacting, diagnosing and the like, or any combination thereof. Manipulations may be performed intrabody, extrabody or both. Manipulations may further be performed within the receptacle cavity, externally to the receptacle cavity, or both.

According to some embodiments there is provided a method of sampling reproductive biological fluid that includes placing a biological fluid sample collector in close proximity to reproductive organs and receiving reproductive biological fluid into a cavity of said biological fluid sample collector, wherein said biological fluid sample collector includes an outer surface having at least two portions with different porosity characteristics, said outer surface further adapted to be affixed to a carrier.

According to some embodiments, the carrier to which the outer surface of the biological fluid sample collector is affixed to may include at least one of the following: contraceptive device, a swab, an applicator, tweezers, tampon, a sampling cup and any combination thereof.

According to some embodiments, the reproductive biological fluids received by the sample collector may be selected from a group that includes semen, seminal fluid, cervical fluid, cervical mucus, vaginal fluid, mammalian cells, microorganisms, proteins, carbohydrates, ions, hormones, or any combination thereof.

According to other embodiments, the reproductive organs may include penis, vagina, cervix and the like and or any combination thereof.

According to further embodiments, at least one outer surface portion with different porosity comprises a membrane.

According to additional embodiments, the biological fluid sample collector cavity may further include inner compartments.

According to some embodiments, the biological fluid sample collector may be used intrabody, extrabody or any combination thereof.

According to further embodiments, the reproductive biological fluids may be further manipulated. Manipulations may include testing, sorting, interacting, diagnosing and the like, or any combination thereof. Manipulations may be performed intrabody, extrabody or both. Manipulations may further be performed within the receptacle cavity, externally to the receptacle cavity, or both.

In addition to the exemplary aspects and embodiments described above, further aspects and embodiments will become apparent by reference to the figures and by study of the following detailed descriptions.

### BRIEF DESCRIPTION OF THE FIGURES

FIG. 1—simplified schematic illustration of a biological fluid receptacle, according to some embodiments;

FIG. 2—perspective view of a receptacle according to some embodiments;

FIG. 3(A-D)—Schematic illustrations of inner cavities of receptacles, according to some embodiments;

FIG. 4(A-C)—perspective view of a receptacle affixed to a male condom, according to some embodiments;

FIG. 5(A-C)—side view of a receptacle affixed to a female sponge, according to some embodiments;

FIG. 6(A-C)—side view of a receptacle affixed to a diaphragm, according to some embodiments;

FIG. 7(A-C)—perspective side view of receptacle, according to some embodiments; and

FIG. 8—perspective side view of receptacle, according to some embodiments.

### DETAILED DESCRIPTION

In the following description, various aspects of the invention will be described. For the purpose of explanation, specific configurations and details are set forth in order to provide a thorough understanding of the invention. However, it will also be apparent to one skilled in the art that the invention may be practiced without specific details being presented herein. Furthermore, well-known features may be omitted or simplified in order not to obscure the invention.

According to some embodiments, there is provided a biological fluid receptacle that may include a receptacle cavity adapted to receive and retain biological fluids. The biological fluid receptacle may be used for the collection of various biological fluids, such as, for example fluids of a mammalian reproductive system and more specifically, fluids of human reproductive system, including but not limited to cells and flora of human reproductive system, such as, for example, semen (sperm cells and seminal plasma), vaginal fluids, cervical fluids, cervical mucus, and the like. The device may be made of a biocompatible material that may include such materials as natural woven or non-woven material and/or a natural or synthetic polymer. The materials may include, for example, such materials as polypropylene, polyvinyldifluoride, polyglycolic acid, polyethylene glycol, silicone, silicone polymer (R₂SiO), methylcellulose, carboxymethylcellulose, hyaluronic acid, nylon, Polyurethane, PTFE, and the like. At least one portion of the device wall may have a higher porosity that may allow penetration of biological fluids such as semen and/or vaginal and/or cervical fluid. Porosity of the receptacle may be determined by various ways, such as, for example: when using a woven material such as silk or nylon, weaving may control porosity; when using other materials, such as synthetic or natural polymers, structural foams like polyurethane foam, latex and any polymer foam, different pore size sectors of the receptacle may be produced, open or closed cell construction, with or without integral skin. The at least one portion of the receptacle wall of a higher porosity can be of the same or different material. When the device is made of a non-woven synthetic polymer or structural foam, the at least one portion of higher porosity may include at least one membrane of same or different material. The membrane may be used to allow selective transfer of biological fluids to the receptacle. The biological fluid receptacle may be placed in close proximity to human reproductive organs, such as penis, vagina, cervix, gonads and the like and the collection of biological fluids may be performed intrabody, for example, during male-female intercourse, or may be placed in a female body for any length of time. The biological fluid receptacle may be further attached/affixed to a carrier, such as a contraceptive device, menstrual device, carrier such as tweezers, swabs and the like that may assist in positioning of the receptacle in a desired location. According to further embodiments, the collection of biological fluids may be performed externally, such as for example, collection of biological fluids (such as semen, sperm cells and seminal plasma) produced as a result of masturbation. The receptacle cavity may further contain internal compartments that may be used for example, for collecting male fluids (such as semen), for collecting female fluids (such as vaginal fluids and cervical mucus), for collecting male and female fluids, and for interacting between male and female fluids (such as, for example in a PCT test). The receptacle may further be used for delivery of various materials and substances that may be present in the device cavity, beforehand the device is filled with the biological fluids. In addition, the receptacle may further be used for holding materials, substances and the like that are capable of nourishment or preservation of cells that may be found in the biological fluids collected and retained in the receptacle cavity. The receptacle may further hold biologically active material that may be capable of treating organ, cells, body, tissue, change vaginal or cervical mucus characteristics, such as, for example, pH, viscosity, volume, that may be favorable of fertilization and may also improve sperm motility/vitality. In addition, the biologically active material may also include hormonal compound, or may include a substance that may be prescribed as treatment or preventive preparation. The biologically active material may thus affect hormonal status.

The biological fluids collected may be further manipulated, such as for example by testing, sorting, diagnosing, and the like. Manipulation of the biological fluids may be performed within the receptacle, both intrabody and extra-body. In addition, the biological fluids collected by the receptacle may be retrieved and manipulated externally (in-vitro), for example by using a diagnostic device and/or in a diagnostic laboratory.

As referred to herein, the term “biological fluid” may include any type of fluid, mucus, secretion and the like (such as blood, plasma, saliva, digestive fluids, cerebrospinal fluid, amniotic fluid, urine, and the like) that may be naturally produced from a living organism, such as, for example, mammalian body, avian body, amphibian body, aquatic-living organism body, and the like. The term “biological fluid” or stimulated form “biological fluid” may further include any substances and flora that the biological fluids may contain, such as, for example, but not limited to: cells, proteins, carbohydrates, ions, hormones, antibodies, polysaccharides, microorganisms, trace elements, and the like. The biological fluid may include, for example, fluids produced by the reproductive system of a mammalian body (“reproductive biological fluids”). The reproductive biological fluids may include, for example: semen and seminal fluids that may include, among others, sperm cells and seminal plasma; vaginal fluids; cervical fluids such as cervical mucus; various cell populations; various associated flora, such as microorganisms, viruses, fungus, parasites; hormones; trace elements; antioxidants; minerals; proteins; carbohydrates; ions; antibodies; polysaccharides, chemicals, and the like.

As referred to herein, the terms “receptacle”, “biological fluid receptacle”, “collecting device”, “biological fluid sample collector” may interchangeably be used.

As referred to herein, the term “sponge” in relation to a female contraceptive device may include any natural foam structure, and/or any artificial structural foam such as polyurethane foam and/or any porous material resembling a sponge that may be used as a contraceptive device.

As referred to herein, the term “porosity characteristics” relates to any type of porosity and/or permeability characteristics, such as pore size, distance between pores, mesh size and the like that may determine the porosity/permeability. According to some embodiments, and as detailed below herein, porosity/permeability characteristics may be different between various region/portions of the biological fluid receptacle.

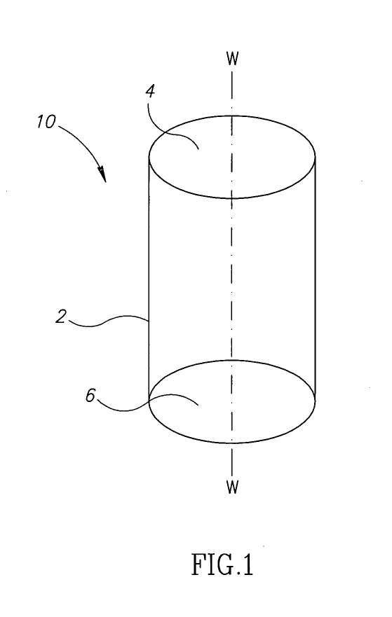

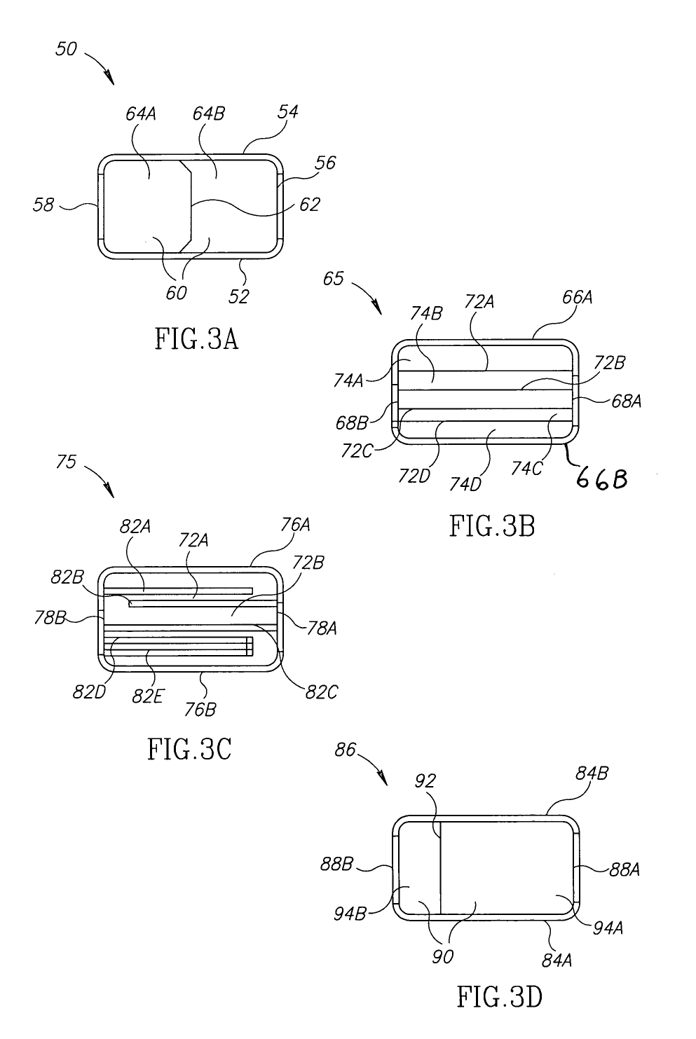

Reference is now made to FIG. 1, which is a simplified schematic illustration of a biological fluid receptacle, according to some embodiments. As schematically shown in FIG. 1, receptacle 10, may have an elongated cylinder-like shape. The receptacle may include outer wall 2, proximal membrane 4 and membrane 6, which is distal to membrane 4. Receptacle 10 may further include an inner cavity, which is enveloped by outer wall 2. Also shown is line w-w, along which various cross section illustrations of receptacle 10 are detailed below herein (in FIG. 3A-D).

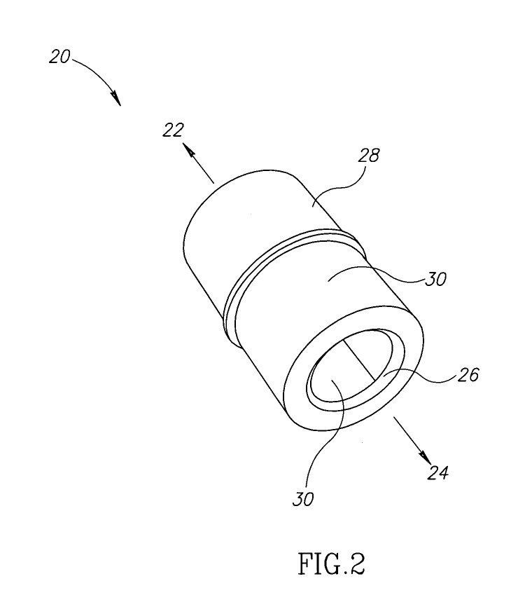

Reference is now made to FIG. 2, which illustrates a perspective view of a biological fluid receptacle, according to some embodiments. Receptacle 20 may have an elongated cylinder-like shape body with at least a partially hollow inner cavity and at least one opposing ends at each verge of the cylinder. The size of the receptacle may vary in length and diameter. For example, the diameter of the receptacle may be in the range of about 6 to 95 mm. For example, the perpendicular height of the receptacle may be in the range of about 1 mil in a shape of a film, to 20 mm in a shape of a cylinder. The external circumference of the receptacle body may be defined by outer walls (28). The outer walls (28) of the receptacle may be smooth and be constructed of biocompatible materials. The outer walls may be comprised of materials that may provide a supporting environment for living cells, so as to help maintain viability of living cells. The outer walls may be comprised of woven and/or non-woven materials. For example, the outer walls may be comprised of such materials as silk, woven silk, Dacron, coated cotton, biocompatible materials used for tissue stitching, natural polymers, synthetic polymers, solid polymers, flexible polymers, polymer films, polymer foams, polypropylene, latex, polyurethane film, polyurethane foam, polyethylene, polyvinyldiflouride, PTFE, polyvinyldiflouride, polyglycolic acid, polyethyleneglycol, silicone, silicone polymers (R₂SiO) methylcellulose, carboxymethylcellulose, hyaluronic acid, nylon and any combination thereof.

The outer walls of the receptacle may be hard, rigid, soft, pressureable, and the like. For example, the softness of the outer walls, as measured in units of shore, may be in the range of, for example, 3 to 45 shore A. The thickness of the outer walls may vary and may be, for example, in the range of about 10 micron to 5000 microns. The outer walls of the receptacle may form one integral continuous body structure. The walls of the receptacle may form two or more continuous body structures that may be permanently or reversibly attached to each other so as to form the body structure of the receptacle. The inner cavity of the receptacle, which is bordered/enveloped by the outer walls of the receptacle, may include one or more compartments/volumes the may be used for example, for the collection, storing, retention, transfer and the like of various fluids. The inner cavity of the receptacle may harbor a total volume of, for example, about 10 to 10000 micro liter. The receptacle may further include two opposing ends at each verge of the receptacle: a male end, 22, and a female end, 24. Each of the ends of the receptacle may be at least partially closed/sealed by various ways, such as by use of cups, plugs, corks, stoppers, septum and the like, that may be extensions of the outer walls, or may be entities that are independent of the outer walls of the receptacle. When the ends of the receptacle are entities/portions of the outer walls, those portions may have different porosity than other portions/regions of the receptacle wall. According to further embodiments, the ends of the receptacle may be at least partially closed/sealed with a membrane. At the verge of either or both ends 22 and/or 24 of the receptacle, a membrane, such as membrane 26 may be situated. The membrane at the male end and female end may be identical or different. The membrane may form as an integral part of the walls of the receptacle, or may be associated with the outer walls of the receptacle. The membranes may be produced integrally with the receptacle walls, or may be pre-produced and assembled to the outer walls of the receptacle. The membrane may be constructed of various materials such as natural polymers, synthetic polymers, nitrocellulose, polycarbonate (such as polycarbonate filter), natural rubber (latex), rubber, silicone, silicon polymers, polyurethane, silk, woven silk, Dacron, coated cotton, polypropylene, polyethylene and any combination thereof. The membranes may further include a mesh surface, a woven surface, a non-woven surface and any combination thereof. The membrane may be further selected from any known and commercially available membranes. The membrane may vary in size and thickness. For example, the thickness of the membrane may be in the range of 1 micron to 2500 micron. The membrane may be held to its location at the end of the receptacle by various ways, such as gluing, adhering, pressure, stitching, heat or ultrasound welding and the like. The membrane may be permanently or reversibly attached to the receptacle body. For example, upon forming the receptacle body, the membrane may be placed to its location and secured by use of biocompatible glue. The membrane may include a semipermeable membrane, which is a membrane that may allow selective transfer of fluid, substances and the like. The size of the pores of the membranes (mesh) may be selected form a variety of ranges, according to the use of the membrane. For example, the membrane may have a pore size of 0.42 nm, (for example, capable of holding hepatitis virus particles) to 200 microns. Portions/regions of the outer walls of the receptacle, which are substantially not permeable, may have pore size of less then 0.42 nm. The transfer of fluids, substances and the like may be unidirectional, for example, from the outer surface of the membrane towards the inner surface of the membrane and hence to the inner cavity of the receptacle. For example, the membrane at the male end may posses such physical and chemical properties so that the transfer of biological fluid, such as, for example, semen (which may include sperm cells) into the inner cavity of the receptacle is allowed. Likewise, the membrane at the female end may poses such physical and chemical properties so that the transfer of biological fluids, such as, for example, vaginal fluids and cervical mucus, into the inner cavity of the receptacle is allowed. In addition, externally to the outer membranes that may be found at the male and/or female ends of the receptacle, gates, such as soluble gates may be positioned. The gates may function as timers that may determine when the membranes may be exposed to the surrounding environment. For example, the gates may include any soluble gates, such as salt gates and sugar gates. When the receptacle is placed in an environment that causes the gates to solubolize, the gates may dissolve and the membranes may be exposed to the surrounding environment. Likewise, hypo-osmolar gates, such as agar gates may be used, that may swell and open under appropriate osmolarity conditions, thus enabling exposure of the membranes to the surrounding environment.

According to some embodiments, an inner cavity, such as 30 of a receptacle, such as 20 may further include one or more compartments/volumes that may be at least partially separated from each other. The inner compartments of the receptacle may be at least partially separated by various ways, such as for example by mechanical means; chemical means, such as different ion concentrations, different pH values; biological means such as enzymes scaffold bio polymers and the like. The inner compartments of the receptacle may be shaped in any shape and may contain any volume. The inner compartments may be separated by various means such as, for example, by inner membranes, gates, barriers, microcapillaries passages, and any combination thereof. For example, the inner compartments may be separated by inner membranes placed at various orientations within the inner compartment of the receptacle. The inner membranes may be identical or different from the outer membranes. For example, the inner compartments may be further separated by gates. The inner compartments may be separated by soluble gates that may include gates that are comprised of any biocompatible soluble material such as sugar gates, salt gates, hypo-osmolar gates, biologically activated gates such as allegenate, lyophilization gates and the like, that may form at least partially separate inner compartments. For example, the inner compartments may be separated by barriers, such as structural barriers that may form at least partially separated inner compartments. For example, the inner compartments may be formed by microcapillaries passages that may run along the at least part of the inner compartment of the receptacle. The micro capillary passages may run at any length and along any axis of the receptacle. For example, the microcapillaries may run along the inner side of the walls of the receptacle, for example, from the male end to the female end. For example, the microcapillaries may run linearly, in spiral shape, serpentine shape and the like.

Reference is now made to FIG. 3, which schematically illustrates exemplary inner cavities of a receptacle, according to some embodiments. FIG. 3A illustrates a cross section of a receptacle, such as receptacle 10 in FIG. 1, according to some embodiments. A two dimensional illustration of a cross section along line w-w of receptacle 10 (FIG. 1) is presented. Outer walls 57 and 54 of receptacle 50 are shown. At the ridge of outer walls 52 and 54, membranes 56 and 58 may be situated. The membranes are located on opposing ends of receptacle 50. Inner cavity, 60, which is bordered by the outer walls 52-54 and membranes 56-58 may be further divided to inner compartments. For example, gate 62 may transverse the inner cavity to create two separate compartments, 64A and 64B. The gate may be used, for example, to separate between two fluids located at compartments 64A and 64B. The gate may further be adapted to open and/or close under various conditions. For example, the gate may be adapted to open and allow fluid transfer between compartments, upon filling of at least one of the compartments. For example, the gate may include a soluble gate, such as a sugar or salt gate that may dissolve upon contact with a fluid entering at least one of the compartments. Opening the gate may allow interaction between fluid in one compartment and fluids/substances in the other compartment. For example, the soluble gate may be used as a timer, to be opened or closed after a required time period (as determined by changes in the solubility properties of the gate) has lapsed. For example, the gate may be comprised of a hypo-osmolar compound, such as, for example, agar, that may be capable of absorbing fluid and/or substances and as a result the gate may be closed and the two compartments be separated such that no transfer of fluids/substances between compartments is allowed. FIG. 3B illustrates a cross section of a receptacle, such as receptacle 10 in FIG. 1, according to some embodiments. A two dimensional illustration of a cross section along line w-w of receptacle 10 (FIG. 1) is presented. Outer walls 66A and 66B of receptacle 65 are shown. At the ridge of outer walls 66A and 66B, membranes 68A and 68B may be situated. The membranes are located on opposing ends of receptacle 65. The inner cavity, which is bordered by the outer walls 66A-B and membranes 68A-B may be further divided to several inner compartments. For example, non-soluble gates, such as gates 72A-D may transverse the inner cavity to create several compartments, such as compartments, 74A-D that are at least partially separated. Compartments thus formed may allow graduate transfer of fluids and substances between adjacent compartments, such as for example, between compartments 74B-D. Further more, some compartments, such as compartment 74A may be used to retain fluids/substances within the compartment and not allow transfer of fluids and/or substances to adjacent compartments. FIG. 3C illustrates a cross section of a receptacle, such as receptacle 10 in FIG. 1, according to some embodiments. A two dimensional illustration of a cross section along line w-w of receptacle 10 (FIG. 1) is presented. Outer walls 76A and 76B of receptacle 75 are shown. At the ridge of outer walls 76A and 76B, membranes 78A and 78B may be situated. The membranes are located on opposing ends of receptacle 75. The inner cavity, which is bordered by the outer walls 76A-B and membranes 78A-B may be further divided to inner compartments by a set of microcapillary passages, such as microcapillaries 82A-E that may run along the inner cavity. The microcapillaries may have any diameter and length and may have one or more openings to allow fluid/substance transfer through the microcapillaries passages. The microcapillaries may include several individual microcapillaries, a branched capillary and any combination thereof. The microcapillary passages may be hollow so as to allow the transfer of fluids/substances within the microcapillaries. In any of the internal volumes/compartments, different environment may be created. Different environment may include, for example, various pH ranges, various osmolarity ranges, various ion content solutions, various substances, molecules, chemical entities, and the like that may reside within the microcapillaries. The various environments within the various microcapillaries may be used, for example, for the separation between various constituents of the biological fluids in the inner compartments. For example, by using various ion contents and application of electrical field on the microcapillaries, substances with different electrical charge may move differently through the microcapillaries channels. Moreover, usage of ion, differential, that may be formed, for example, by different soluble salt mixtures may induct an electrical field that may be used for the separation between various constituents of the biological fluids in the inner compartments. FIG. 3D illustrates a cross section of a receptacle, such as receptacle 10 in FIG. 1, according to some embodiments. A two dimensional illustration of a cross section along line w-w of receptacle 10 (FIG. 1) is presented. Outer walls 84A-B of receptacle 86 are shown. At the ridges of outer walls 84A-B, membranes 88A-B may be situated. The membranes may be located on opposing ends of receptacle 86. Inner cavity, 90, which is bordered by the outer walls 84A-B and membranes 88A-B may be further divided to inner compartments. For example, an additional, inner membrane (92) may transverse the inner cavity, and thus create two compartments, 94A-B. The inner membrane properties, such as pore size may be used to determine fluid/substance transfer between compartments 94A-B. The inner membrane(s) may be similar or different that the outer membranes.

According to some embodiments, the biological fluid receptacle may have an amorphous shape. The amorphous shaped receptacle may be comprised of outer walls that may be flexible and may form at least a partially closed compartment, which may create an inner cavity. The inner cavity of the receptacle may harbor a total volume of, for example, about 50 to 6000 micro-liter. The outer walls (outer surface) of the receptacle, or various portions/regions of the receptacle walls may, according to some examples, function as semipermeable membranes that allow a selective transfer of fluids and substances into the cavity of the receptacle. According to other embodiments, the walls of the receptacle may be essentially non-permeable and as such they may have pore size, which is smaller 0.42 nm. The outer walls may be comprised of woven and/or non-woven materials. For example, the outer walls may be comprised of such materials as silk, woven silk, Dacron, coated cotton, biocompatible materials used for tissue stitching, natural polymers, synthetic polymers, solid polymers, flexible polymers, polymer films, polymer foams, polypropylene, latex, polyurethane film, polyurethane foam, polyethylene, polyvinyldiflouride, PTFE polyvinyldiflouride, polyglycolic acid, polyethyleneglycol, silicone, silicone polymers (R₂SiO) methylcellulose, carboxymethylcellulose, hyaluronic acid, nylon and any combination thereof. The receptacle may further include one or more membranes that may be embedded in the walls of the receptacle. The membranes may be semipermeable membranes that may allow a selective transfer of fluids and substances into the inner compartment of the receptacle. The membranes may be constructed of a mesh, woven material, non-woven material and any combination thereof. For example, the membranes may be constructed of various materials such as natural polymers, synthetic polymers, nitrocellulose, polycarbonate (such as polycarbonate filter), natural rubber (latex), rubber, silicone, silicone polymers, polyurethane, PTFE, silk, woven silk, Dacron, coated cotton, polypropylene, polyethylene and any combination thereof. The membranes may form as an integral part of the receptacle walls, wherein the region at which the membranes are located may exhibit porosity and/or permeability properties that are different than the essentially non permeable properties of further regions/portions of the outer walls. For example, the membranes may include a mesh or woven surface that may form as integral part of the receptacle walls and may function as semipermeable barrier. The membranes may be selected from any known and commercially available membranes. The membranes may vary in size and thickness. For example, the thickness of the membranes may be in the range of 1 micron to 2500 micron. The membranes may be held to its location at the receptacle by various ways, such as gluing, adhering, pressure, stitching, heat or ultrasound welding and the like. The membranes may be permanently or reversibly attached to the receptacle. For example, upon forming the receptacle body, the membranes may be placed to its location and secured by use of biocompatible glue. The membranes may include a semipermeable membrane, which is a membrane that may allow selective transfer of fluid, substances and the like. The size of the pores of the membranes (mesh) may be selected from a variety of ranges, according to the use of the membrane. For example, the membrane may have a pore size of 0.42 nm to 200 microns. The transfer of fluids, substances and the like may be unidirectional, for example, from the outer surface of the membrane towards the inner surface of the membrane and hence to the inner cavity of the receptacle. The inner cavity of the receptacle may further include one or more sub-compartments that may be at least partially separated from each other and may allow selective transfer and storage of various fluids, such as, for example, biological fluids. The fluids may include, for example, semen, seminal fluid, cervical mucus, vaginal fluids and the like. The inner cavity may further include additional constituents, such as supporting medium, substances, buffers and any other biologically relevant material. The additional constituents may reside within the receptacle cavity compartments beforehand the receptacle is used and filled with biological fluids. For example, the additional constituents may be used to support viability of cells of the biological fluids, such as for example, sperm cells, cervical cells and the like. For example, the additional constituents may be used for further manipulations of the biological fluids as further detailed below herein.

According to other embodiments, the receptacle may be used to collect biological fluids, such as fluids produced by the human reproductive system. For example, fluids produced by the male, such as semen may be collected at the male end of the receptacle. Fluids produced by the female, such as vaginal and cervical fluids may be collected at the female end of the receptacle. The biological fluids may be collected intrabody (within the body, such as for example, within the genital/reproductive tracts of a female) and/or extra-body (externally to a body). For example, the receptacle may be used intrabody during male-female intercourse and may thus collect biological fluids produced by the male and/or from the female. According to additional examples, the receptacle may be placed intrabody in the female genital tracts for any number of hours, such as in the range of 0.5-48 hours. Thus, the receptacle may collect biological fluids, such as vaginal and cervical fluids produced by the female. In addition, the biological fluid receptacle may also be used extra-body. For example, the biological fluid receptacle may be placed/associated with an external carrier, such as a sampling cup. In this setting, biological fluids, such as, for example, semen produced by masturbation and ejaculated into the sampling cup may be collected by the receptacle.

According to additional embodiments, and as exemplified above herein, the various inner compartments that may reside within the inner cavity of the receptacle, may be identical or different in size, shape and inner content. The inner compartments may be used for various purposes. For example, the inner compartments may be used for the separation between various fluids and substances collected within the receptacle cavity. The biological fluids produced by the male and/or female may each be contained in separate compartments within the receptacle. The use of various compartment settings may further allow manipulations of the female and/or male biological fluids collected within the receptacle. Manipulations may include, for example, testing, sorting, interacting, diagnosing, and the like of the male and female biological fluids and substances contained therein. The testing may include, for example, various biologically related parameters, such as sperm cells concentration, sperm cells morphology and cell count, that may be present in the biological fluids; Sperm related parameters, such as sperm cells count, sperm cells volume, sperm cells motility, progressively motile sperm cells, sperm cells vitality and the like. Testing of the fluids may further include testing parameters such as pH levels, presence of specific antibodies, white blood cells concentration, protein composition, carbohydrates, lectins, fructose, zinc and the like. Testing may also include microbiological related parameters, such as detecting presence of fungus, parasites, bacteria, viruses and the like. In addition, testing may further include any of the WHO standards (1999) for the examination of human semen including: semen appearance, semen volume, semen viscosity, semen pH, sperm cells concentration, sperm cells motility, cellular elements other than spermatozoa, sperm cells agglutination, sperm cells vitality, sperm cells morphology, antibody-coating of spermatozoa, hypo-osmotic swelling test, semen culture, biochemical assays for accessory sex organ function (zinc, fructose and neutral α-glucosidase), computer-aided sperm analysis, zona-free hamster oocyte test, reactive oxygen species, human zona pellucida binding test, acrosome reaction, anti oxidants and trace elements.

Sorting may include sorting various cell populations, sorting of viable sperm cells, and the like. Diagnosing may include any diagnostic test that may be used to determine, for example, male/female infertility related test, such as, sperm cell number, sperm cells motility, specific sperm cells antibodies, and the like. Diagnosing may further include any diagnostic test that may be used to determine health related conditions of male and/or female. For example, health related conditions may include disease conditions that may be caused by various infections, such as, for example: Syphilis, Gonorrhea, Candida Albicans, Human Papilloma Virus (HPV), Mycoplasma Hominis, Ureaplasma Urealyticum, Human Immunodeficiency Virus (HIV), Condiloma Acunimado, Trichomonas Vaginalis, Group B Sterptococcus (GBS) infection, Gardnerella Vaginalis, Chlamydia Trachomatio and the like. For example, health related conditions may include disease conditions such as cancer (prostate cancer, cervix cancer, pre-cancer cells and the like). Interaction testing may be used, for example to test any interaction parameters between the male reproductive fluids, such as for example, sperm cells and the female reproductive fluids.

The manipulations may be performed within the receptacle, by aid and use of various biologically relevant buffers, mediums, substances and the like, that may be present within various designated compartments of the receptacle, beforehand or afterhand the receptacle has been filled with biological fluids. The biologically relevant buffers, substances, mediums and the like that may be used for manipulations of the biological fluids may include, for example, but not limited to: sperm cells chemoattractants that are capable of guiding or enhancing sperm cells motility, such as, for example: glycoaminoglycan hyaluronic acid (HA) which has been shown to increase spermatozoa motility; progesterone, when present in follicular fluid, causes accumulation and activation of spermatozoa; Atrial natriuretic peptide (ANP) is a polypeptide hormone that is secreted from different cell types in humans, is an activator of particulate guanylate cyclase that attracts spermatozoa and increase speed of motility; Heparin was found as a constitutive component of follicular fluid that also causes accumulation and activation of spermatozoa; Bourgeonal (4-t-butylbenzenepropionaldehyde) acts as a strong chemoattractant in sperm cells behavioral assays; Chemokines with direct effect of on the function of human sperm cells were reported; RANTES (Regulated on Activation Normal T Expressed and Secreted Chemokine) is a protein, member of the interleukin-8 superfamily of cytokines, found in genital tract fluids (follicular fluid, seminal plasma and uterine fluid) and acts as a chemoattractant on spermatozoa. Additional biological substances may include Hyaluronic acid containing mediums; Various antibodies directed against various epitopes, such as, for example, anti CD-59 antibody; Various staining reagents, such as tiazin based staining agents, toloidin blue, and the like that may be used according to known protocols. In addition, the biologically relevant buffers, substances, mediums and the like may be used to provide a supporting environment for cells that may be found in biological fluids of the human reproductive system.

Manipulation of biological fluids collected within the receptacle may be performed by various ways and at various possible settings. As mentioned aboveherein, manipulations of the biological fluids may be performed in the receptacle device cavity, for example, within various compartments of the receptacle cavity. The manipulation of the biological fluids within the receptacle cavity may be performed intrabody and/or extra-body. According to other examples, manipulation of the biological fluids may be performed externally to the receptacle. For example, the biological fluids may be transferred by various ways from the receptacle into an external diagnostic device. The external diagnostic device may include, for example, a home based diagnostic device that may be operated by a home user and be used to test the biological fluids samples. In addition, the biological fluids may be transferred by various ways from the receptacle to an external location, such as a diagnostic laboratory, wherein various manipulations of the biological fluids may be performed.

According to some embodiments, the biological fluid receptacle may be manufactured by various methods. The methods may include such techniques as, injection mould, rotational mould, press mould, dipping mould, spin cast, structural foam mould, weaving techniques and any other suitable manufacturing method. For example, a mould may be used to prepare a receptacle body, receptacle membranes, receptacle inner compartments and any combination thereof. For example, a mould may be used to prepare a receptacle with integral membrane. For example, the membrane may be placed in a mould. The membrane may be impregnated with protecting, soluble compounds, any compound of solvent-soluble material, and the like, such as, for example water-soluble compounds like sugar and salt. Into the mould, a mixture of materials that may comprise the receptacle body (outer walls) may be added. Such compounds may include any woven and/or non-woven material, such as, for example, silk, woven silk, Dacron, coated cotton, biocompatible materials used for tissue stitching, natural polymers, synthetic polymers, solid polymers, flexible polymers, polymer films, polymer foams, polypropylene, latex, polyurethane film, polyurethane foam, polyethylene, polyvinyldiflouride, PTFE polyvinyldiflouride, polyglycolic acid, polyethyleneglycol, silicone, silicone polymers (R₂SiO) methylcellulose, carboxymethylcellulose, hyaluronic acid, nylon and any combination thereof. Upon solidification of the receptacle walls, the soluble compound that impregnated the membrane may be removed, for example, by solubolizing the soluble compound. Thus, the membrane physical and mechanical properties may be exposed again, without being affected by the molding of the receptacle outer walls. According to additional example, the membrane may be held inside a mould by a ram and anvil. Into the mould, a mixture of materials that may comprise the receptacle body (outer walls) may be added. Nevertheless, the pores of the membrane may be protected by the ram and anvil and thus, upon solidification of the receptacle walls and removal of the ram and anvil, the membrane pores may be exposed again. According to additional examples, a structural foam mould may be used to manufacture a receptacle. The method of structural foam molding, which is well known in the art, may produce structural products, which contain elements with different physical and mechanical properties. The method may generally include injection of a structural foam compound into a mould. For example this method may be used to prepare closed or open constructions with integral walls with various pore size. This may be achieved, for example by controlling the thermal gradient in the ramparts of the mould that may influence the foam properties and hence type of product that may be produced. In addition, control of venting of the mould internal volume, during injection of the structural foam into the mould may create encapsulated internal volume within the product. For example, for the manufacturing of a receptacle using the method of structural foam mold, thermal gradient may be used to control the type of the walls of the receptacle, the thickness of the walls of the receptacle, the porosity of the walls, and the like. In addition, control of venting of the mould internal volume, while the structural foam is being injected into the mold may create internal cavity (and/or sub-compartments) within the receptacle.

According to some embodiments, the biological fluid receptacle may further be associated/affixed to a carrier that may be used to carry and position the receptacle to a desired location. For example, the carrier may be used to position the receptacle intrabody, at a desired location, in close proximity to reproductive organs and gonads, such as penis, vagina, cervix, and the like. In such a setting, reproductive system biological fluids produced, for example, during intercourse, may be collected intrabody by the receptacle. Likewise, cervical fluids produced routinely by a female may be collected intrabody by a receptacle that may be placed within the female body for a time period of, for example, 0.5 to 48 hours. Carriers that may be used to position the receptacle intrabody may include such carriers as: male contraceptive devices (for example, a condom); female contraceptive devices (for example, sponge, diaphragm, female condom, cervical cap, and the like); various applicators capable of being positioned inside the vagina such as swabs, sticks, handles, tweezers; any menstrual device such as tampon, menstrual flow, and the like. The receptacle may be permanently or reversibly affixed/associated to the carrier. Associating between the receptacle and the carrier may be performed by various ways, such as for example, use of biocompatible materials, adhering, stitching, heat or ultrasound welding, melting, dipping, dip molding, pressuring, mechanical means and any other known suitable method. According to further embodiments, the carrier may be used to position the receptacle at a location that is external to the body. In such a setting biological fluids of the reproductive system may be collected, for example by masturbation. Fluids, such as semen produced by ejaculation may be collected by the receptacle that may be placed in or on an external carrier, such as for example, a sampling cup.

According to additional embodiments, the biological fluid receptacle may be associated/affixed to a contraceptive device. The contraceptive device may include a male contraceptive device, such as a male condom. The contraceptive device may include a female contraceptive device, such as a diaphragm, cervical cap, sponge, female condom and the like. The receptacle may be permanently associated with the contraceptive device. The receptacle may be temporarily associated with the contraceptive device. The receptacle may be associated with the contraceptive device by being placed inside the contraceptive device. The receptacle may be associated with the contraceptive device by being attached to the surface of the contraceptive device. Association (affixing) between the receptacle and the contraceptive device may be performed by various ways and methods, such as for example, by gluing, adhering, stitching, use of pressure, heat or ultrasound welding, melting, dipping, dip molding, and the like. Associating between the receptacle and the contraceptive device may be performed such that the receptacle may form an integral part of the contraceptive device. For example, the receptacle may be embedded as part of the routine preparation method of the contraceptive device. For example, the contraceptive device and the receptacle may be molded together to form one integral unit. Associating between the receptacle and the contraceptive device may be performed such that the receptacle is attached/affixed by any know method, to a fully assembled, ready for use, contraceptive.

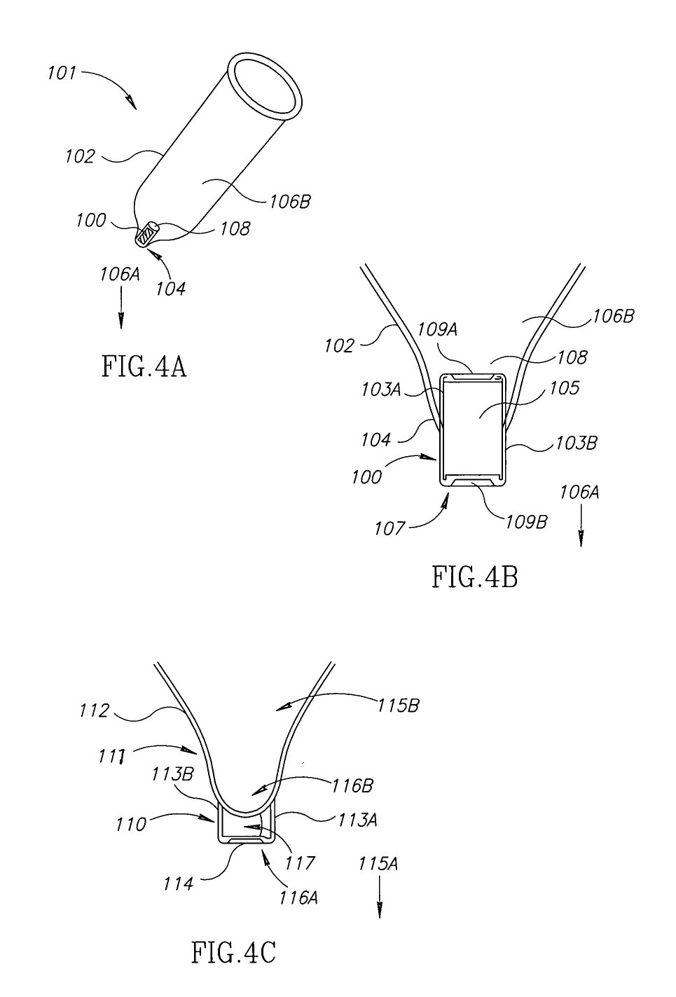

According to some embodiments, the receptacle may be associated with a male condom, as illustrated by way of example in FIG. 4A. As shown in FIG. 4A, a receptacle, (such as 100) may be placed inside a male condom, such as 102, in close proximity to the tip (closed end, 104) of the condom. The receptacle (100) may be permanently attached to the male condom 102, for example by stitching, adhering, heat or ultrasound welding, melting, dipping, dip molding and the like. The male condom (102) may be manufactured such that the condom is molded around the receptacle, so that the receptacle is located at the tip of the condom at the completion of the manufacturing process. The receptacle (100) may transverse the tip (104) of the condom such that the female end (not shown) of the receptacle is located on the outer side (106A) of the condom and the male end (108) of the receptacle is located within the inner portion (106B) of the condom. For example, an opening at the tip of condom 102 may be made. Into the opening, receptacle 100 may be placed such that the female end (not shown) of the receptacle is located on the outer side (106A) of the condom and the male end (108) of the receptacle is located within the inner portion (106B) of the condom. The contact region between the opening in the condom and the outer surface (outer walls) of the receptacle may be sealed by various methods, such as gluing, stitching, heat or ultrasound welding, melting, dipping, dip molding and the like. Reference is now made to FIG. 4B, which illustrates a close up view of a receptacle transversing the tip of a condom, according to some embodiments. As shown in FIG. 4B, receptacle (such as, 100) may transverse the tip (104) of the condom such that the female end (107) of the receptacle is located on the outer side (106A) of the condom and the male end (108) of the receptacle is located within the inner portion (106B) of the condom. Outer membranes, such as membranes 109A and 109B may be located at the extremities of the receptacle, at the male end (108) and the female end (107), respectively. The outer membranes may be attached to the outer walls (103A-B) of the receptacle by various ways, as detailed above herein and may be constructed of various materials such as natural polymers, synthetic polymers, nitrocellulose, polycarbonate (such as polycarbonate filter), natural rubber (latex), rubber, silicone, silicone polymers, polyurethane, PTFE, silk, woven silk, Dacron, coated cotton, polypropylene, polyethylene and any combination thereof. The membranes may further include a mesh surface, a woven surface, a non-woven surface and any combination thereof. The membrane may also be selected from any known and commercially available membranes. The outer membranes may form as an integral part of the receptacle walls, wherein the region/portion at which the membranes are located may exhibit porosity and/or permeability properties that are different than the essentially non permeable properties of further regions of the outer walls of the receptacle. For example, the outer membranes may include a mesh or woven surface. Cavity (such as 105) of the receptacle may be used for reception and retention of biological fluids. As detailed above herein with reference to FIG. 3, cavity 105 may further be divided to internal sub-compartments by various ways, such as for example, internal gates, internal membranes that may be similar or different to outer membranes 109A-B, capillaries, and the like. Cavity 105 may further be divided into sub-compartments by, for example a septum, that may include an essentially non-permeable barrier such as the condom walls, that may define two separate sub-compartments, a male end facing sub compartment and a female end facing sub compartment.

According to some embodiments, the receptacle-associated condom (such as 101) may be used intra body, for example, during male-female intercourse. Biological fluids produced during and as a result of the intercourse may be collected by the receptacle in such a way that male fluids, such as semen may be collected though the male end (108) of the receptacle and the female fluids, such as cervical fluids and vaginal fluids may be collected at the female end of the receptacle. In addition, the receptacle-associated condom (101) may be used extra body, to collect biological fluids produced externally, such as, for example, during masturbation The biological fluids collected within the cavity of the receptacle (100) may be contained within compartments of the inner cavity, as detailed above herein. The biological fluids may further be manipulated, as detailed above herein, internally to the receptacle, externally to the receptacle, intrabody, extra body and in any combination thereof.

According to some embodiments, and as further illustrated in the FIG. 4C, which illustrates a close up view of a receptacle associated with a condom, according to some embodiments, a receptacle (such as 110) may be attached to a male condom (such as, 112). Receptacle 110 may include an outer membrane, such as membrane (114) on one end (female end, 116A) and an essentially non-permeable barrier at the opposing end (male end, 116B). The outer membrane (114) may be attached to the outer walls (113A-B) of the receptacle by various ways, as detailed above herein and may be constructed of various materials such as natural polymers, synthetic polymers, nitrocellulose, polycarbonate (such as polycarbonate filter), natural rubber (latex), rubber, silicone, silicone polymers, polyurethane, PTFE, silk, woven silk, Dacron, coated cotton, polypropylene, polyethylene and any combination thereof. The membranes may further include a mesh surface, a woven surface, a non-woven surface and any combination thereof. The membrane may be further selected from any known and commercially available membranes. The outer membrane may form as an integral part of the receptacle walls, wherein the region at which the membrane is located may exhibit porosity and/or permeability properties that are different than the essentially non permeable properties of further regions of the outer walls. For example, the outer membrane may include a mesh or woven surface. Cavity 117 of the receptacle may be used for reception and retention of biological fluids, such as for example, cervical/vaginal mucus, cervical/vaginal fluids and the like. As detailed above herein with reference to FIG. 3, cavity 117 may further be divided to internal sub-compartments by various ways, such as for example, internal gates, internal membranes that may be similar or different to the outer membrane, capillaries, and the like. The receptacle 110 may be attached to the tip of the condom on the external side (115A) of the condom. Receptacle (110) may be permanently attached to the male condom 112, for example by stitching, adhering, heat or ultrasound welding, melting, dipping, dip molding and the like.

According to some embodiments, the receptacle-associated condom (such as 111) may be used during male-female intercourse. Biological fluids produced during and as a result of the intercourse may be collected by the receptacle in such a way that female fluids, such as cervical fluids and vaginal fluids may be collected at the female end of the receptacle, which is located on the external side (115A) of condom 112. Male fluids produced during and as a result of the intercourse may be collected in the condom, at the inner side (115B) of the condom (112). The female biological fluids collected within the cavity of the receptacle may be contained within compartments of the inner cavity, as detailed above herein. The biological fluids collected may further be manipulated, as detailed above herein, internally to the receptacle, externally to the receptacle, intrabody, extra body and in any combination thereof.

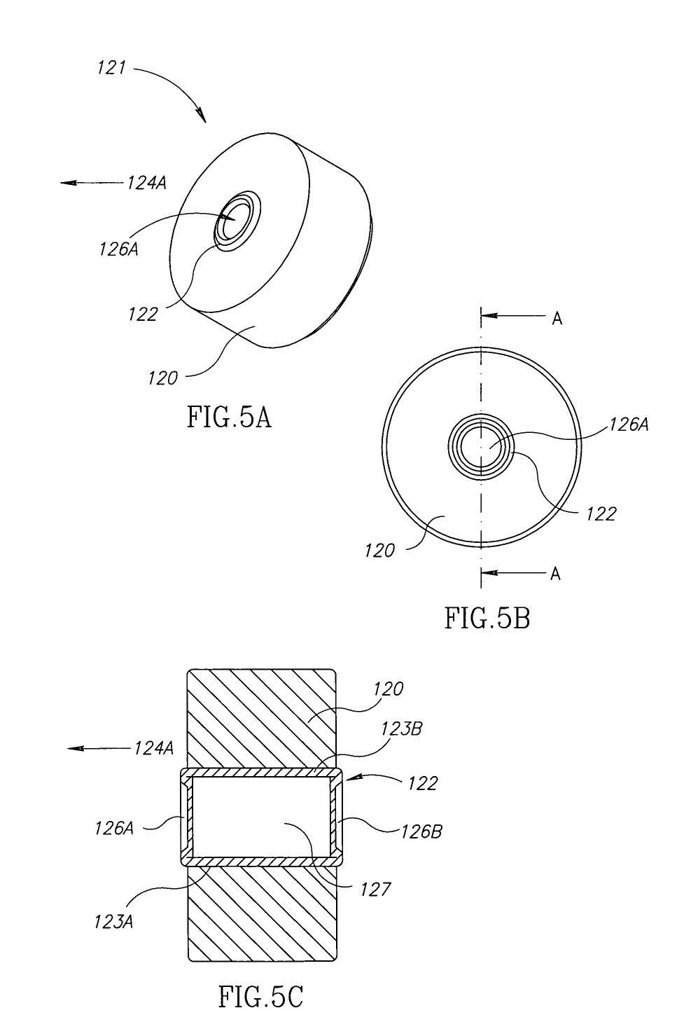

According to some embodiments, the receptacle may be associated with a female sponge contraceptive device. A female sponge is a commercially available contraceptive device that may include a disposable, disc-shaped, usually comprised of polyurethane foam, which may cover the cervix and thus function as a contraceptive. FIG. 5 illustrates a receptacle associated with a sponge, according to some embodiments. Reference is now made to FIG. 5A, which illustrates a perspective view of a receptacle attached to a sponge, according to some embodiments. As shown in FIG. 5A, a round, disc-shaped sponge contraceptive device (such as 120) is provided. At approximately the center of the sponge, a receptacle device (such as 122) may be located. The sponge may include an opening at its center that may fit the receptacle. The receptacle may be secured to its location in the sponge by various ways, such as by stitching, adhering, heat or ultrasound welding, melting, dipping, dip molding and the like. The receptacle may form as an integral part of the sponge. The receptacle may transverse the sponge from side to side, along the central axis of the sponge such that the ends of the receptacle (for example, female end, 124A) and male end (not shown) are located on opposite faces of the sponge. At one or more ends of the receptacle, one or more outer membranes may be located. For example, membrane 126A, shown in FIG. 5A, may face the female end (124A). Likewise, a similar or different membrane may be located at the opposing male end. Outer membrane, such as membrane 126 may include any semipermeable membrane that may allow a selective transfer of biological fluids to the inner cavity of the receptacle. Outer membrane, such as membrane 126A may be attached to the receptacle walls by various ways, as detailed above herein, and may be constructed of various materials such as natural polymers, synthetic polymers, nitrocellulose, polycarbonate (such as polycarbonate filter), natural rubber (latex), rubber, silicone, silicone polymers, polyurethane, PTFE, silk, woven silk, Dacron, coated cotton, polypropylene, polyethylene and any combination thereof. The membranes may further include a mesh surface, a woven surface, a non-woven surface and any combination thereof. The membrane may be further selected from any known and commercially available membranes. The outer membrane may form as an integral part of the receptacle walls, wherein the portion/region at which the membrane is located may exhibit porosity and/or permeability properties that are different than the essentially non permeable properties of further portions/regions of the outer walls. The cavity of the receptacle, which is defined by the receptacle borders (walls and membranes) may be used for reception and retention of biological fluids, such as for example, cervical/vaginal mucus, cervical/vaginal fluids and the like. As detailed above herein with reference to FIG. 3, the cavity may further be divided to internal sub-compartments by various ways, such as for example, internal gates, internal membranes that may be similar or different to the outer membrane, capillaries, and the like. FIG. 5B illustrates a top view of the female end of a receptacle associated with a sponge, according to some embodiments. Shown in FIG. 5B a round, sponge contraceptive device sponge (such as 120) and at approximately the center of the sponge, a receptacle (such as 122) may be located. The sponge may include an opening at its center that may fit the receptacle. The receptacle may be secured to its location in the sponge by various ways, such as by stitching, adhering, heat or ultrasound welding, melting, dipping, dip molding and the like. The receptacle may transverse the sponge from side to side, along the central axis of the sponge such that the ends of the receptacle are located on opposite faces of the sponge. At one or more ends of the receptacle, one or more outer membranes, such as membrane 126A may be located. FIG. 5C illustrates a side view of a cross section of a receptacle associated with a sponge, taken along line A-A in FIG. 5B. As shown in FIG. B, at the center of sponge (such as 120), a receptacle (such as 122) is located. Receptacle 122 may transverse the sponge from side to side, along the central axis of the sponge such that the ends of the receptacle (for example, female end, 124A and male end 124B) are located on opposite faces of the sponge. At one or both ends of the receptacle, one or more outer membranes may be located. The outer membranes, such as membranes 126A and 126B may be located at the extremities of the receptacle, at the female end (124A) and the male end (124B), respectively. The outer membranes may be attached to the outer walls (123A-B) of the receptacle by various ways, as detailed above herein and may be constructed of various materials such as natural polymers, synthetic polymers, nitrocellulose, polycarbonate (such as polycarbonate filter), natural rubber (latex), rubber, silicone, silicone polymers, polyurethane, PTFE, silk, woven silk, Dacron, coated cotton, polypropylene, polyethylene and any combination thereof. The membranes may further include a mesh surface, a woven surface, a non-woven surface and any combination thereof. The membrane may be selected from any known and commercially available membranes. The outer membranes may also form as an integral part of the receptacle walls, wherein the portion/region at which the membranes are located may exhibit porosity and/or permeability properties that are different than the essentially non permeable properties of further portions/regions of the outer walls. Cavity 127 of the receptacle may be used for reception and retention of biological fluids. As detailed above herein with reference to FIG. 3, cavity 127 may further be divided to internal sub-compartments by various ways, such as for example, internal gates, internal membranes that may be similar or different to outer membranes 126A-B, capillaries, and the like.

According to some embodiments, the receptacle-associated sponge (121) may be used intrabody during male-female intercourse. The receptacle-associated sponge (121) may be inserted to its location within the vaginal tract (covering the cervix) of a female subject, prior to the intercourse. Biological fluids produced during and as a result of the intercourse may be collected by the receptacle in such a way that male fluids, such as semen may be collected at the male end of the receptacle and the female fluids, such as cervical fluids and vaginal fluids may be collected at the female end (124A) of the receptacle. In addition, the receptacle-associated sponge may be used intrabody by being inserted to a location in the female genital tracts for any number of hours, such as in the range of 0.5-48 hours. The biological fluids collected within the cavity of the receptacle may be contained within compartments of the inner cavity of the receptacle, as detailed above herein. The biological fluids may further be manipulated, as detailed above herein, internally to the receptacle, externally to the receptacle, intrabody, extra body and in any combination thereof.

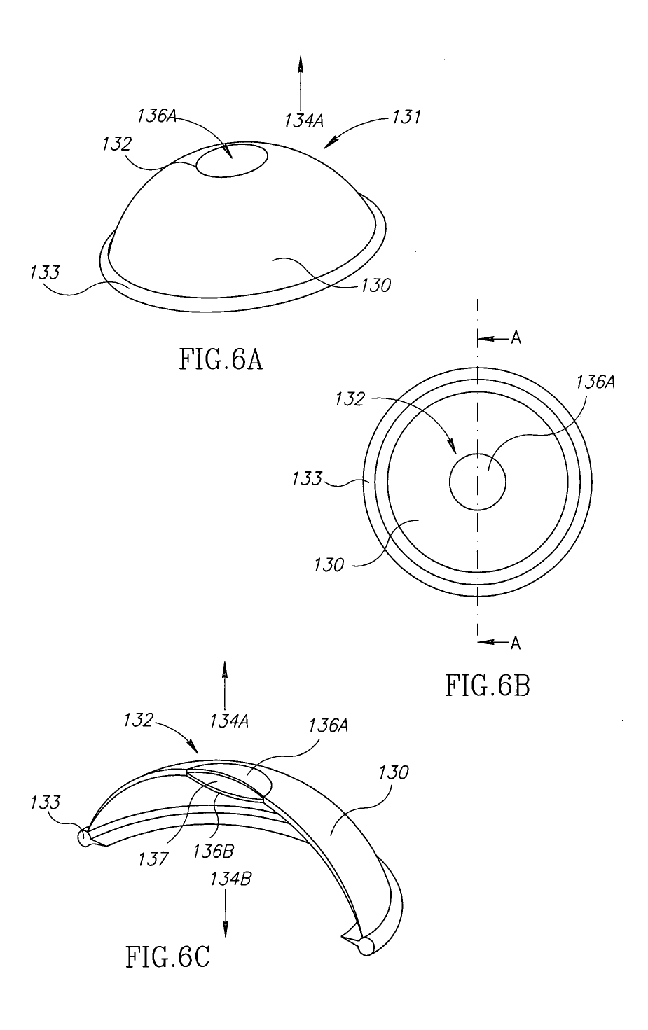

According to some embodiments, the receptacle may be associated with a female diaphragm. A female diaphragm is a dome-shaped disk that may be inserted into the vagina before intercourse. The diaphragm may be comprised of, for example, a soft latex or silicone dome with a spring, or provide a spring-like action molded into the rim. The spring/spring-like action may be used to create a seal against the walls of the vagina at the back of the cervix, against the pubic bone. The rim of a diaphragm may be squeezed into an oval or an arc or an eight shape for insertion. Reference is now made to FIG. 6A, which illustrates a perspective view of a receptacle attached to a diaphragm (131), according to some embodiments. As shown in FIG. 6A, a dome-shaped diaphragm contraceptive device (such as 130) is provided. The rim (such as 133) at the outer circumference of the diaphragm may include a spring or provide a spring-like action. At approximately the center of the arched-dome-region of the diaphragm, a receptacle device (such as 132) may be located. The diaphragm may include an opening at the center of the arched-dome region that may fit the receptacle. The receptacle may be secured to its location in the diaphragm by various ways, such as by stitching, adhering, heat or ultrasound welding, melting, dipping, dip molding and the like. The receptacle may form as an integral part of the diaphragm. The receptacle may transverse the diaphragm from side to side, along the central axis of the diaphragm dome such that the ends of the receptacle (for example, male end, 134A) and female end (not shown) are located on opposite faces of the diaphragm. For example, the female end may protrude to the concaved surface of the diaphragm and the male end may protrude to the arched surface of the diaphragm. At one or more ends of the receptacle, one or more outer membranes may be located. For example, outer membrane 136A may face the male end (134A). Likewise, a similar or different membrane may be located at the opposing female end. Outer membrane, such as outer membrane 136A may include any semipermeable membrane that may allow a selective transfer of biological fluids to the inner cavity of the receptacle. Outer membrane, such as membrane 136A may be attached to the receptacle walls by various ways, as detailed above herein, and may be constructed of various materials such as, for example, natural polymers, synthetic polymers, nitrocellulose, polycarbonate (such as polycarbonate filter), natural rubber (latex), rubber, silicone, silicone polymers, polyurethane, PTFE, silk, woven silk, Dacron, coated cotton, polypropylene, polyethylene and any combination thereof. The membranes may further include a mesh surface, a woven surface, a non-woven surface and any combination thereof. The membrane may be selected from any known and commercially available membranes. The outer membrane may further form as an integral part of the receptacle walls, wherein the region/portion at which the membrane is located may exhibit porosity and/or permeability properties that are different than the essentially non permeable properties of further regions/portions of the outer walls. The cavity of the receptacle, which is defined by the receptacle borders (walls and membranes) may be used for reception and retention of biological fluids, such as for example, cervical/vaginal mucus, cervical/vaginal fluids and the like. As detailed above herein with reference to FIG. 3, the cavity may further be divided to internal sub-compartments by various ways, such as for example, internal gates, internal membranes that may be similar or different to the outer membrane, capillaries, and the like. FIG. 6B illustrates a top view of the male end of a receptacle associated with a diaphragm, according to some embodiments. Shown in FIG. 6B a round, diaphragm contraceptive device (such as 130) with a rim (such as 133) at the external circumference of the diaphragm, and at approximately the center of the diaphragm, a receptacle (such as 132) may be located. The diaphragm may include an opening at its center that may fit the receptacle. The receptacle may be secured to its location in the diaphragm by various ways, such as by stitching, adhering, heat or ultrasound welding, melting, dipping, dip molding and the like. The receptacle may further form as an integral part of the diaphragm. The receptacle may transverse the diaphragm from side to side, along the central axis of the diaphragm such that the ends of the receptacle are located on opposite faces of the diaphragm. At one or more ends of the receptacle, one or more outer membranes, such as membrane 136A may be located. FIG. 6C illustrates a side view cross section of a receptacle associated with a diaphragm, taken along line A-A in FIG. 6B. As shown in FIG. 6C, at about the center of the arched dome region of the diaphragm (such as 130), a receptacle (such as 132) may be located. Receptacle 132 may transverse the diaphragm from side to side, along the central axis of the diaphragm such that the ends of the receptacle (for example, male end, 134A and female end 134B) are located on opposite faces of the diaphragm. At one or both ends of the receptacle, one or more outer membranes may be located. The outer membranes, such as membranes 136A and 136B may be located at the extremities of the receptacle, at the male end (134A) and the female end (134B), respectively. The outer membranes may be attached to the outer walls of the receptacle by various ways, as detailed above herein and may be constructed of various materials such as, for example, natural polymers, synthetic polymers, nitrocellulose, polycarbonate (such as polycarbonate filter), natural rubber (latex), rubber, silicone, silicone polymers, polyurethane, PTFE, silk; woven silk, Dacron, coated cotton, polypropylene, polyethylene and any combination thereof. The membranes may further include a mesh surface, a woven surface, a non-woven surface and any combination thereof. The membrane may be selected from any known and commercially available membranes. The outer membranes may form as an integral part of the receptacle walls, wherein the region/portion at which the membranes are located may exhibit porosity and/or permeability properties that are different than the essentially non permeable properties of further regions/portions of the outer walls. Cavity 137 of the receptacle may be used for reception and retention of biological fluids. As detailed above herein with reference to FIG. 3, cavity 137 may further be divided to internal sub-compartments by various ways, such as for example, internal gates, internal membranes that may be similar or different to outer membranes 136A-B, capillaries, and the like. Cavity 137 may further be divided into sub-compartments by, for example a septum, that may include an essentially non-permeable barrier such as diaphragm walls, that may define two separate sub-compartments, a male end facing sub-compartment and a female end facing sub-compartment.

According to some embodiments, the receptacle-associated diaphragm (131) may be used intrabody during male-female intercourse. The receptacle-associated diaphragm (131) may be inserted to its location within the vaginal tract of a female subject, prior to the intercourse, for example by squeezing into an oval or an arc or an eight shape for insertion and positioning over the cervix. Biological fluids produced during and as a result of the intercourse may be collected by the receptacle (130) in such a way that male fluids, such as semen and seminal fluids may be collected at the male end (134B) of the receptacle and the female fluids, such as cervical fluids and vaginal fluids may be collected at the female end, of the receptacle (130). In addition, the receptacle-associated diaphragm may be used intrabody by being inserted to a location in the female genital tracts for any number of hours, such as in the range of 0.5-48 hours. The biological fluids collected within the cavity of the receptacle may be contained within compartments of the inner cavity, as detailed above herein. The biological fluids may further be manipulated, as detailed above herein, internally to the receptacle, externally to the receptacle, intrabody, extra body and in any combination thereof.

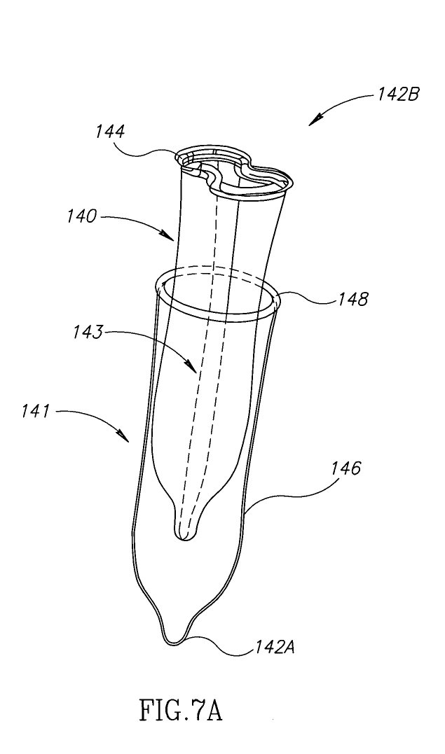

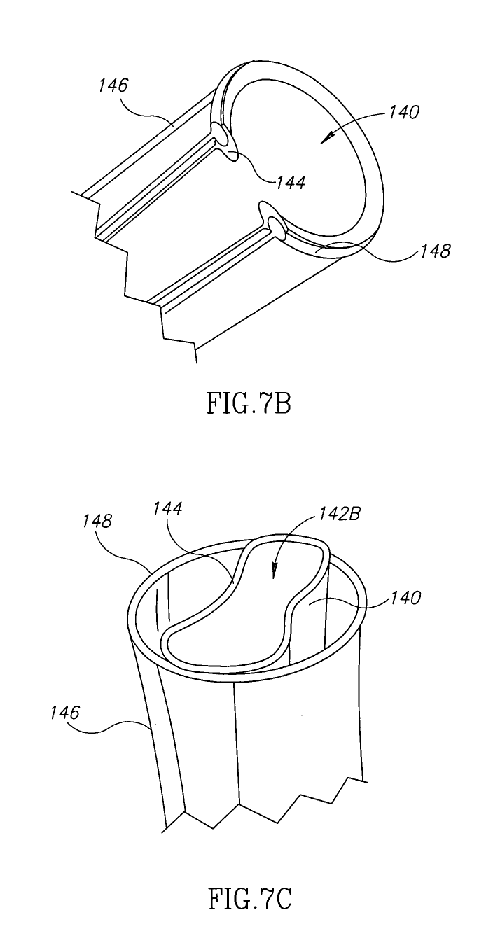

Reference is now made to FIG. 7, which illustrates a receptacle, according to some embodiments. As shown in FIG. 7A, which illustrates a perspective side view of a receptacle according to some embodiments, the receptacle (such as 140) may include an elongated, flexible bag, which may have a condom-like shape with inner cavity (such as 143) defined by the walls of the receptacle bag (140). The narrow end (142A) of the receptacle may be at least partially sealed. For example, the walls at the narrow end of the receptacle may exhibit pore sizes of less then 0.42 nm. The broad end (142B), which is distally opposing the narrow end (142A), may be open. The broad, open end (142B) may further include a rim (144) that may be used for the association of the receptacle within a contraceptive, as detailed below herein. Rim 144 is illustrated in FIG. 7A at a deformed state, wherein the rim is shaped into an eight form, by, for example, pinching two opposing sides of the rim towards each other. The receptacle (140) may be constructed of rubber, silk, polyurethane, silicone and the like. The thickness of the receptacle may vary in the range of about 5 to 1500 microns. Size of receptacle 140 may vary in length and diameter. For example, length of receptacle 140 from end 142A to end 142B may be in the range of, about 100-200 mm. For example, diameter of rim 144 of receptacle 140 may be in the range of, about 37 to 60 millimeter. Receptacle (such as 140) may further be associated with a male contraceptive device, such as a condom 146. Receptacle 140 may be fitted into male condom 146, such that the receptacle is contained within the inner space of the condom. Shown in FIG. 7A is a receptacle 140 fitted about two thirds of its length into a condom 146. Receptacle 140 may be secured to condom 146 by various ways. For example, rim 144 and rim 148 of condom 146 may be associated by pressing, stitching, mechanical fitting, zip-lock fit, zipper fit, fitting grooves, adhering, gluing and the like. For example, rim 144 of receptacle 140 may include perforation/grooves that may be used to fit to the upper rim, 148 of condom 146. An example of fitting of rim 144 of receptacle 140 onto rim 148 of condom 146 is illustrated in FIG. 7B, which illustrates a close-up view of rims 144 and 148. As illustrated in FIG. 7B, the rim (such as 144) of receptacle 140 may clamp and encircle the rim (such as 148) of condom 146 in a manner which is similar to a zip-lock fitting. By fitting protrusions located along rim 148 into grooves in rim 144, receptacle 140 may be hanged on rim 148 of condom 146. Removal of receptacle 140 from condom 146 may be performed by for example, deforming the shape of receptacle 140, such that it may be separated from condom 146. For example, and as illustrated in FIG. 7C, the broad end (142B) of receptacle 140 may be accessed and rim 144 may be deformed be pressing/pinching the rim edges towards each other. Upon deformation of receptacle 140 it may be removed from condom 146 and the content of the receptacle may be collected. Receptacle, such as receptacle 140 may be manufactured individually, and then assembled into a premade condom. Receptacle, such as receptacle 140 may be manufactured individually and a spin cast may be used for manufacturing the outer condom on the already made receptacle. Receptacle, such as receptacle 140 may be manufactured integrally with a male condom. Receptacle, such as receptacle 140 may be manufactured is association with a male condom, such as for example, by rolling the condom and the receptacle together.

According to some embodiments, the receptacle-associated condom (141), respectively) may be used intra body during male-female intercourse. Biological fluids produced during and as a result of the intercourse may be collected by the receptacle (140) in such a way that male fluids, produced during and as a result of the intercourse may be collected in the cavity (143) of the receptacle (140). Also, female fluids may be collected on the outer surface of condom 146. In addition, the receptacle associated condom may be used extrabody, to collect biological fluids produced externally, such as, for example, during masturbation. The biological fluids thus collected may further be manipulated, as detailed above herein, internally to the receptacle, externally to the receptacle, intrabody, extrabody and in any combination thereof.

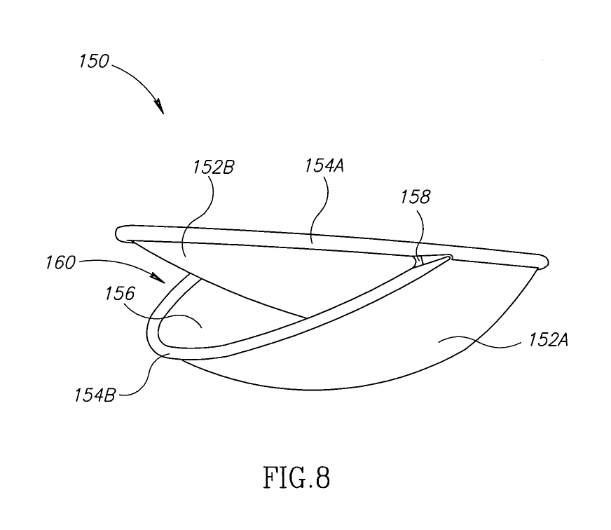

Reference is now made to FIG. 8, which illustrates a perspective side view of a receptacle according to some embodiments. As shown in FIG. 8, the receptacle (such as 152B) may be associated with a contraceptive device, such as a diaphragm to acquire a two-lipped dome-like shaped structure (such as, 150). Structure 150 may be comprised of at least two parts (152A and 152B) that may acquire a dome-like structures that may be at least partially connected/integrated. Part 152A may include a contraceptive, such as a diaphragm (and may thus acquire a diaphragm-like structure with an extended rim, 154A along the outer circumference of the diaphragm. Part 152A may be comprised of, for example, a soft polyurethane latex or silicone dome. Part 152B, may function as a receptacle and may acquire a diaphragm-like structure with an extended rim, 154B, along the outer circumference of the diaphragm dome-like structure. Part 152B may be identical or different in size, form and construction material to part 152A. Part 152B may be constructed of, for example, silk, various polymers such as polyurethane, rubber, latex, silicone, and the like. Part 152B may be fitted to part 152A such that the concaved side of part 152B encircles/covers the arched surface of part 152A. The fitting between parts 152A and 152B may be such that a cavity (such as 156) may be formed between the concaved side of part 152B and the arched surface of part 152A. Parts 152A and 152B may be tightly associated at their respective rims, 154A and 154B, at least along half of the rims circumference, after which, that rims may separate and distant from each other. At the point of separation between rims 154A and 154B, a hinge point, such as hinge point 158 may be formed, and at the region where rims 154A and 154E are not in close contact, an opening 160 may be formed. The opening (160) thus formed may acquire a lip-like structure, the borders of which may be defined by unassociated rims 154A and 154B. The opening (160) may further be at least partially covered with a semipermeable membrane that may allow selective transfer of fluids and substances into cavity 156. According to some embodiments, parts 152A and 152B may be manufactured separately by routine manufacturing procedures, such as by use of moulds and then assembled by connecting their respective rims along at least part of the outer circumferences of the rims. Connecting the rims may be performed by various methods, such as gluing, adhering, heat or ultrasound welding, stitching, mechanical fitting (such as zipper fit, zip-lock fit, fitting grooves), pressing, and the like. Parts 152A and 152B may also be manufactured as one integral unit, for example by use of specifically designed mould. In this way, the two parts may share a common rim at least along part of the circumference of the rim, and then the rims may split such that each part may acquire an individual rim.

According to some embodiments, the receptacle-associated diaphragm (such as 150) may be used intrabody, during male-female intercourse. The receptacle-associated diaphragm may be inserted to its location within the vaginal tract of a female subject, prior to the intercourse by, for example, squeezing the receptacle-associated diaphragm (150) into an oval or an arc or an eight shape for insertion and positioning. The receptacle-associated diaphragm (150) may be placed such that the concaved surface of part 152A may face the female cervix. Receptacle part 152B and opening 160 may thus face the male side. Biological fluids produced during and as a result of the intercourse may be collected by the receptacle in such a way that male fluids, such as semen and seminal fluid may enter cavity 156 through opening 160 and be further collected in cavity 156. Female fluids, such as cervical fluids and vaginal fluids may be collected at the concaved region of diaphragm part 152A. In addition, the receptacle may be used intrabody by being inserted to a location in the female genital tracts for any number of hours, such as in the range of 0.5-48 hours. The biological fluids thus collected may further be manipulated, as detailed above herein, internally to the receptacle, externally to the receptacle, intrabody, extra body and in any combination thereof.

According to some embodiments, there are also provided methods for testing male fertility. Methods for testing male infertility may include such methods as described in provisional application (U.S. Provisional application No. 60/845,173), incorporated herein by reference. For example a method for testing male fertility may include acquiring a sample of the seminal fluid and sperm cells collected in the biological fluid receptacle and testing in said sample the sperm cells number and/or the sperm cells motility and/or progressive motile sperm cells and/or sperm cells vitality and/or semen volume, and/or chemotaxis directed motility, and/or the compatibility of sperm cells sample to the cervical mucus of a female, and any other diagnostic test usually performed for diagnosis of male/female infertility such as, but not limited to, pH level, sperm cells antibodies, fungus, parasites, bacteria, protein composition, carbohydrates, Fructose concentration, Zinc concentration, and/or cell populations such as white blood cells, erythrocytes, epithelial cells, and lymphocytes. For example testing of seminal fluids and sperm cells collected in the receptacle may further include any of the WHO standards (1999) for the examination of human semen including such tests as: semen appearance, semen volume, semen viscosity, semen pH, sperm cells concentration, sperm cells motility, cellular elements other than spermatozoa, sperm cells agglutination, sperm cells vitality, sperm cells morphology, antibody-coating of spermatozoa, hypo-osmotic swelling test, semen culture, biochemical assays for accessory sex organ function like zinc, fructose, neutral α-glucosidase, computer-aided sperm analysis, zona-free hamster oocyte test, reactive oxygen species, human zona pellucida binding test, acrosome reaction, anti oxidants and trace elements. Testing the biological fluids may be performed in the receptacle device cavity, for example, within various compartments of the receptacle cavity. Testing performed within the receptacle device may be performed intrabody and/or extra-body. According to other examples, testing of the biological fluids may be performed externally to the receptacle. For example, the biological fluids may be transferred by various ways from the receptacle into an external diagnostic device. The external diagnostic device may include, for example, a home based diagnostic device that may be operated by a home user. In addition, the biological fluids may be transferred by various ways from the receptacle and be tested in a diagnostic laboratory, using standard protocols and procedures.

The method for testing male infertility may further allow detection of parameters of sperm cells quality such as: concentration, motility, progressive motility, directed motility and vitality. Also, the method may be capable of detection of compatibility of sperm cells to cervical mucus or cervical fluid. In one embodiment, this method may be used for detection of all said parameters in natural way, intrabody, during, for example coitus. With regard to the factors that are tested regarding male infertility is to be understood that: (i) motility—low motility of sperm cells is explained by abnormal morphology and/or problems with energy production capacity and/or vitality; (ii) chemotaxis—chemotaxis-directed motility allows fertilization of the egg by morphologically normal sperm cells; and (iii) compatibility, i.e., compatibility of sperm cells to female cervical mucus.

In addition, female biological fluids, such as cervical mucus and vaginal fluids, that may be collected by the biological fluid receptacle, may also be tested, for example by the postcoital test (PCT) to imply infertility issues. This test, also known as the cervical mucus penetration test, is designed to evaluate the effect of a woman's cervical mucus on a man's sperm cells. Typically, a woman is asked to come into the physician's office within two to 24 hours after intercourse at mid-cycle (when ovulation should occur). A small sample of her cervical mucus is examined under a microscope. If the physician observes no surviving sperm cells or no sperm cells at all, the cervical mucus should then be cultured for the presence of infection. The test cannot evaluate sperm cells movement from the cervix into the fallopian tubes or the sperm cells' ability to fertilize an egg. Additional tests that can be carried in the cervical mucus include pH level, sperm cells antibodies, fungus, parasites, bacteria, protein composition, carbohydrates, and cell populations such as: white blood cells, erythrocytes, epithelial cells and lymphocytes. Also, sperm cells penetration into human cervical mucus in vitro is known to provide important predictive information about sperm cells function. Testing the female biological fluids may be performed in the receptacle device cavity, for example, within various compartments of the receptacle cavity. Testing performed within the receptacle device may be performed intrabody, and/or extra-body. According to other examples, testing of the biological fluids may be performed externally to the receptacle. For example, the biological fluids may be transferred by various ways from the receptacle into an external diagnostic device. The external diagnostic device may include, for example, a home based diagnostic device that may be operated by a home user. In addition, the biological fluids may be transferred by various ways from the receptacle and be tested in a diagnostic laboratory by using standard procedures and protocols.

According to additional embodiments, the biological fluid receptacle may be used to collect biological fluids from various organisms other than human. For example, the receptacle may be used for the collection of biological fluids, from organisms such as equines, cattle, swines, pedigree dogs, sus scrofa, turkeys and domestic fowls the like. The collected biological fluids may include, for example, semen (such as seminal plasma and sperm cells) and the like. Collection of the biological fluids may be used, for example, to improve artificial insemination (a process by which sperm are collected from the male, processed, stored and artificially introduced into the female reproductive tract for the purpose of conception), which is an important techniques used in the for the genetic improvement of animals. The biological fluid receptacle may be used intrabody and/or extrabody to collect biological fluids. For example, the receptacle (that may be associated/affixed to a carrier, as detailed above herein) may be placed intrabody within the genital tract of a mammalian female (such as a mare or a cow) prior to mating with a male (such as an equine or a bull, respectively). Thus, biological fluids produced during mating, such as semen may be collected by the receptacle device. According to additional examples, the receptacle may be used extra body by being placed/associated within an external carrier, such as a sampling cup, into which biological fluids, such as, semen of an equine, bull and the like may be collected. The biological fluids of the organisms other than human, such as equines, cattle, swines, pedigree dogs, sus scrofa, turkeys and domestic fowls, and the like may further be manipulated. As detailed above herein, manipulations may include, for example, testing, sorting, interacting, diagnosing, and the like of the biological fluids. For example, manipulation of biological fluids, such as semen from equines may be used to determine the quality and efficacy of the semen samples. Such tests may include, for example test to determine diseases or other health related issues of the equine, such as: Glanders: intradermo-palpebral test or complement fixation test; Equine infectious anemia: immuno diffusion test; Equine piroplasmosis: complement fixation test; Contagious equine metritis: bacteriological culture from reproductive organs in at least 3 tests at intervals of at least 7 days; Equine viral arteritis: serum neutralization test; Salmonellosis: tube agglutination test; Vesicular stomatitis: serum neutralization test; (such as described in: “Quarantine Requirements for the Importation of Equine Semen”, Promulgated by Council of Agriculture on Nov. 26, 1986, First amendment by Council of Agriculture on Apr. 19, 1994, incorporated herein by reference) and any other disease and diagnostic tests that may be designated. Manipulations of the biological fluids may be performed, for example within the receptacle, either intrabody or extrabody. Manipulation of the biological fluids collected from mammals such as equines and cattle, may be performed externally to the receptacle. For example, the biological fluids may be transferred by various ways from the receptacle into an external diagnostic device. In addition, the biological fluids may be transferred by various ways from the receptacle and be tested in a diagnostic laboratory.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

What we claim is:

1. A biological fluid sample collector comprising:
an outer surface adapted to be affixed to a carrier and comprising at least two portions with different porosity characteristics; and
a sample collector cavity adapted to receive biological fluids.

2. The biological fluid sample collector of claim 1, wherein said carrier comprises at least one of the following: contraceptive device, a swab, an applicator, tweezers, menstrual device, tampon, a sampling cup.

3. The biological fluid sample collector of claim 1, wherein said biological fluid is selected from a group consisting of: semen, seminal fluid, cervical fluid, cervical mucus, vaginal fluid, mammalian cells, microorganisms, proteins, carbohydrates, ions, hormones, antibodies, toxins, chemicals or any combination thereof.

4. The biological fluid sample collector of claim 1, wherein at least one outer surface portion with different porosity comprises a membrane.

5. The biological fluid sample collector of claim 1, wherein said biological fluid collector cavity further comprises inner compartments.

6. The biological fluid sample collector of claim 1, wherein said biological fluid sample collector is used intrabody, extrabody or any combination thereof.

7. The biological fluid sample collector of claim 1, wherein said biological fluids are further manipulated.

8. The biological fluid sample collector of claim 7, wherein said manipulation comprises testing, sorting, interacting, diagnosing, or any combination thereof.

9. The biological fluid sample collector of claim 8 wherein said manipulation is performed intrabody, extrabody or both.

10. The biological fluid sample collector of claim 8 wherein said manipulation is performed within the biological fluid sample collector cavity, externally to the biological fluid sample collector cavity, or both.

11. A biological fluid receptacle comprising:
an outer surface adapted to be affixed to a contraceptive device and comprising at least two portions with different porosity characteristics; and
a receptacle cavity adapted to receive biological fluids.

12. The biological fluid receptacle of claim 11 wherein the biological fluid is selected from a group comprising: semen, seminal fluid, cervical fluid, cervical mucus, vaginal fluid, mammalian cells, microorganisms, proteins, carbohydrates, ions, hormones, antibodies, toxins, chemicals or any combination thereof.

13. The biological fluid receptacle of claim 11, wherein said contraceptive device is selected from a group comprising: male condom, sponge, diaphragm, cervical cup, female condom or any combination thereof.

14. The biological fluid sample collector of claim 11, wherein at least one outer surface portion with different porosity comprises a membrane.

15. The biological fluid receptacle of claim 11, wherein said receptacle cavity further comprises inner compartments.

16. The biological fluid receptacle of claim 11, wherein said biological fluid receptacle is used intra body, extrabody or any combination thereof.

17. The biological receptacle of claim 11, wherein said biological fluids are further manipulated.

18. The biological fluid sample collector of claim 17, wherein said manipulation comprises testing, sorting, interacting, diagnosing or any combination thereof.

19. The biological receptacle of claim 17 wherein said manipulation is performed intrabody, extrabody or both.

20. The biological receptacle of claim 17 wherein said manipulation is performed within the receptacle cavity, externally to the receptacle cavity, or both.

21. A biological fluid receptacle comprising:
an outer surface integral with a contraceptive device; and
a receptacle cavity adapted to receive biological fluids.

22. The biological fluid receptacle of claim 21, wherein said biological fluid is selected from a group comprising: semen, seminal fluid, cervical fluid, cervical mucus, vaginal fluid, mammalian cells, microorganisms, proteins, carbohydrates, ions, hormones, antibodies, toxins, chemicals and any combination thereof.

23. The biological fluid receptacle of claim 21, wherein the contraceptive device is selected from a group comprising: male condom, diaphragm, cervical cup, female condom or any combination thereof.

24. The biological fluid receptacle of claim 21, wherein said biological fluid receptacle is used intra body, extrabody or any combination thereof.

25. The biological receptacle of claim 21, wherein said biological fluids are further manipulated.

26. The biological fluid sample collector of claim 25, wherein said manipulation comprises testing, sorting, interacting, diagnosing, or any combination thereof.

27. The biological receptacle of claim 26 wherein said manipulation is performed intrabody, extrabody or both.

28. The biological receptacle of claim 26 wherein said manipulation is performed within the receptacle cavity, externally to the receptacle cavity, or both.

29. A method of sampling reproductive biological fluid comprising:
placing a biological fluid sample collector in close proximity to reproductive organs; and
receiving reproductive biological fluid into a cavity of said biological fluid sample collector, wherein said biological fluid sample collector includes an outer surface having at least two portions with different porosity characteristics, said outer surface further adapted to be affixed to a carrier.

30. The method of claim 29, wherein said carrier comprises at least one of the following: contraceptive device, a swab, an applicator, tweezers, tampon, a sampling cup.

31. The method of claim 29, wherein said reproductive biological fluid is selected from a group consisting of: semen, seminal fluid, cervical fluid, cervical mucus, vaginal fluid, mammalian cells, microorganisms, proteins, carbohydrates, ions, hormones, antibodies, toxins, chemicals or any combination thereof.

32. The method of claim 29, wherein said reproductive organs include penis, vagina, cervix, or any combination thereof.

33. The method of claim 29, wherein at least one outer surface portion with different porosity comprises a membrane.

34. The method of claim 29, wherein said biological fluid sample collector cavity further comprises inner compartments.

35. The method of claim 29, wherein said biological fluid sample collector is used intrabody, extrabody or any combination thereof.

36. The method of claim 29, wherein said reproductive biological fluids are further manipulated.

37. The method of claim 36, wherein in said manipulation comprises testing, sorting, interacting, diagnosing, or any combination thereof.

38. The method of claim 36, wherein said manipulation is performed intrabody, extrabody or both.

39. The method of claim 36 wherein said manipulation is performed within the biological fluid sample collector cavity, externally to the biological fluid sample collector cavity, or both.
